(19) United States
(12) Patent Application Publication
(10) Pub. No.: US 2020/0297287 A1
(43) Pub. Date: Sep. 24, 2020

(54) SYSTEM AND METHOD FOR AUTOMATED RULES BASED ASSESSMENT OF ANEURYSM COIL STABILITY

(71) Applicant: The Board of Regents of the University of Texas System, Austin, TX (US)

(72) Inventors: Christopher R. Conner, Houston, TX (US); Peng R. Chen, Houston, TX (US)

(21) Appl. No.: 16/814,283

(22) Filed: Mar. 10, 2020

Related U.S. Application Data
(60) Provisional application No. 62/821,152, filed on Mar. 20, 2019.

(51) Int. Cl. A61B 5/00; A61B 17/12; G16H 10/60; G16H 50/30; G16H 50/20; G16H 30/40

(52) U.S. Cl. CPC A61B 5/7275; A61B 17/12113; A61B 5/7264; G16H 30/40; G16H 50/30; G16H 50/20; G16H 10/60

(57) ABSTRACT

Embodiments of systems and methods for rules based assessment of endovascular coil stability are disclosed. These systems and methods may assess images from a patient taken at a first time, such as a time of initial coil placement or thereafter, and at one or more second times, using a set of rules. Based on this assessment, a predictive recurrence indicator predictive of recurrence of the coil in the patient may be determined and presented to a user of the system (such as a doctor or other clinician). This predictive recurrence indicator can then be utilized to determine if further procedures should be undertaken to diagnose or treat the patient.

### RELATED APPLICATIONS

This application claims a benefit of priority to the filing date of U.S. Provisional Patent Application Ser. No. 62/821,152 filed on Mar. 20, 2019, entitled “SYSTEM AND METHOD FOR AUTOMATED RULES BASED ASSESSMENT OF ANEURYSM COIL STABILITY,” by inventors Conner and Chen, the entire contents of which are hereby expressly incorporated by reference for all purposes.

### COPYRIGHT NOTICE

A portion of the disclosure of this patent document contains material to which a claim for copyright is made. The copyright owner has no objection to the facsimile reproduction by anyone of the patent document or the patent disclosure as it appears in the Patent and Trademark Office patent file or records but reserves all other copyright rights whatsoever.

### TECHNICAL FIELD

This disclosure relates generally to the field of medical informatics. More specifically, the disclosure relates to embodiments of systems and methods for the rules based computerized analysis of patient related data in a medical setting. Even more particularly, embodiments of this disclosure relate to the automated rules based analysis of patient data to evaluate the stability of endovascular aneurysm coils.

### BACKGROUND

An estimated 6 million people in the United States have an unruptured brain aneurysm, or about 1 in 50 people. The annual rate of rupture of these aneurysms is approximately 8-10 per 100,000 people or, put another way, about 30,000 people in the United States suffer a brain aneurysm rupture every year. To put it more starkly, there is a brain aneurysm rupturing every 18 minutes. These ruptured brain aneurysms are fatal in about 40% of cases and, of those who survive, about 66% suffer some permanent neurological deficit.

The treatment of these aneurysms typically takes one of two approaches: endovascular coiling or surgical clipping. Endovascular coiling of aneurysms has grown significantly over the past decade, with a majority of aneurysms now treated by coiling as opposed to open surgery for clipping. Typically, a coiling procedure is performed as an extension of an angiogram. A catheter is inserted into a vessel over the hip and other catheters are navigated through the blood vessels to the vessels of the brain and into the aneurysm. Coils (e.g., usually a metallic material such as platinum) are then packed into the aneurysm up to the point where it arises from the blood vessel, preventing blood flow from entering the aneurysm. The long-term success of endovascular coiling to treat aneurysms is about 80 to 85%.

The treatment durability of coiling is, however, still under study, with aneurysm recurrence of approximately 16-40% requiring re-treatment in about 10-20% of cases. Recurrence happens if the coils do not completely block off the aneurysm or if the coils become compacted within the aneurysm. If a major portion of the aneurysm remains unfilled, additional coils or a surgical clip can be placed to stop the growth. A recurrence may, however, not be significant enough to require additional treatment.

Because the risk of aneurysm recurrence after endovascular coiling is higher than surgical clipping, all patients with coiled aneurysms are usually advised to return after some period of time (e.g., 6, 12, and 24 months) for a diagnostic evaluation to monitor for a residual or recurring aneurysm. The current standard to ensure treatment durability, and to prevent devastating cerebral hemorrhage from recurrence, is catheter-based angiography. However, follow up with catheter angiography is both invasive (risking clinically significant embolic strokes, hematomas and vascular injury) and expensive, requiring serial studies costing thousands of dollars.

Alternatives to evaluation using catheter based angiography have included computerized tomography (CT) or magnetic resonance (MR) based angiography, but these techniques are usually inaccurate due to the presence of metallic coil mass artifacts and difficulty in assessing for recurrence measuring less than about 3 mm. Furthermore, CT or MR angiography is expensive and not available to all patient populations.

While in certain cases, x-rays have been used by doctors to assess the stability of endovascular coils, this assessment is performed in a manual and individually subjective assessment by that doctor based on that particular doctor's experience and knowledge.

What is desired, then, are automated systems and methods for assessing the stability of endovascular coils using relatively inexpensive and readily available imaging techniques such as x-rays or the like.

### SUMMARY

To that end, among others, embodiments of systems and methods for rules based assessment of endovascular coil stability are disclosed herein. Embodiments of these systems and methods may assess images from a patient taken at a first time (e.g., at the time of initial coil placement or thereafter) and at one or more second times (e.g., at 6, 12 or 24 months from coil placement) using a set of rules. Based on this assessment, a predictive recurrence parameter or indicator (e.g., a yes or no, a likelihood or probability of recurrence, etc.) may be determined and presented to a user of the system (such as a doctor or other clinician). This predictive recurrence predictor or indicator (used interchangeably herein) can then be utilized to determine if further procedures should be undertaken to diagnose or treat the patient.

The rules utilized by embodiments of the systems and methods may be generated based on an analysis of previously obtained patient data. In particular, a corpus of patient data may include a set of patient records for patients who have had a coiling procedure performed. These patient records each include a set of images (e.g., x-rays) for that patient (e.g., taken at least at a first and second time) and a recurrence indicator indicating whether that patient has a recurrence of their aneurysm subsequently to having the coiling procedure.

This patient data can be analyzed to generate the rule set to be utilized. Specifically, in certain embodiments one or more metrics of the coil may be determined for each patient in the corpus (or a subset of those patients that may comprise a training data set or the like) based on the set of images for that patient. These coil metrics may include metrics related to properties of the coil such as density or dimensions (e.g., length, width or area) of the coil or the coils relation to other structures (e.g., its relation to bony structures of the body). These coil metrics determined for each of the patients may be utilized along with the recurrence indicator for that patient to determine the rule set utilized by embodiments of the systems and methods as disclosed.

In one embodiment an aneurysm coil assessment system, may operate to obtain patient data on a set of patients, each patient having had an aneurysm coil procedure for an aneurysm, wherein the patient data for a first patient includes a first image of a coil placed in the aneurysm coil procedure for the first patient from a first time and a second image of the coil of the first patient from a second time and a recurrence indicator indicating whether that first patient has a recurrence of their aneurysm subsequently to having the aneurysm coiling procedure.

The aneurysm coil assessment system can generate values for a set of coil assessment metrics (e.g., density of the coil, length of the coil, or width of the coil, etc.) for each of the set of patients from the patient data for each patient, wherein the values for the set of coil assessment metrics for the first patient is based on the first image of the coil of the first patient from the first time and the second image of the coil of that first patient for the second time. Based on the values for the set of coil assessment metrics for each of the set of patients and the recurrence indicator associated with each of the set of patients a rule set for generating a predictive recurrence indicator can be generated.

At some point the aneurysm coil assessment system may receive an indication from a user that a predictive recurrence indicator is to be generated for a second patient and obtain a first image of a coil placed in an aneurysm coiling procedure for the second patient from a first time and a second image of the coil of the second patient from a second time. Values for the set of coil assessment metrics for the second patient can be generated based on the first image of the coil of the second patient from the first time and the second image of the coil of that second patient for the second time and the rule set applied to the values for the set of coil assessment metrics for the second patient to generate a predictive recurrence indicator based on the values for the set of coil assessment metrics for the second patient. The predictive recurrence indicator predictive of recurrence of the coil in the second patient can be presented to the user.

The images for the second patient may, for example, be obtained from an imaging device over a computing network, such as an x-ray machine providing an anterior-posterior skull x-ray or a lateral skull x-ray.

In some embodiments, generating values for the set of coil assessment metrics for each of the set of patients comprises aligning the first image of the coil of the first patient from the first time with the second image of the coil of that first patient for the second time and generating the values for the set of coil assessment metrics for the second patient comprises aligning the first image of the coil of the second patient from the first time with the second image of the coil of that second patient for the second time.

In particular embodiments, aligning the first image and the second image for the first patient comprises aligning the coil in the first image of the first patient with the coil in the second image of the first patient and aligning the first image and the second image for the second patient comprises aligning the coil in the first image of the second patient with the coil in the second image of the second patient.

Thus, embodiments as disclosed may have a number of advantages. As a particular advantage to embodiments of these systems and methods, the images used for assessment need not be from a catheter angiogram, a CT scan, or an MR scan. Instead, a simple and usually readily available and relatively inexpensive x-ray may be utilized for one or both the images at the first and the second point in time.

In this way, the x-ray (or other image) comparison and predictive recurrence indicator will potentially be able to prevent the need for a follow up angiogram in certain patients, eliminating the risks or disadvantages associated with any catheter angiogram procedure, which include embolic strokes, hematomas, vascular injury, and cost (e.g., angiograms are expensive averaging about $3,500 a procedure versus a $100 x-ray).

Additionally, the automated rules based assessment of coil stability may allow the simultaneous consideration of multiple variables including, for example, the dimensions (length, width or area) and density of the coil when generating a single metric (or score) of recurrence. Moreover, the analysis may be automated, requiring no clinician involvement while also being adapted to be continually improved through the generation of new rules based on better or additional patient data.

These, and other, aspects of the disclosure will be better appreciated and understood when considered in conjunction with the following description and the accompanying drawings. It should be understood, however, that the following description, while indicating various embodiments of the disclosure and numerous specific details thereof, is given by way of illustration and not of limitation. Many substitutions, modifications, additions and/or rearrangements may be made within the scope of the disclosure without departing from the spirit thereof, and the disclosure includes all such substitutions, modifications, additions and/or rearrangements.

### BRIEF DESCRIPTION OF THE DRAWINGS

The drawings accompanying and forming part of this specification are included to depict certain aspects of the invention. A clearer impression of the invention, and of the components and operation of systems provided with the invention, will become more readily apparent by referring to the exemplary, and therefore nonlimiting, embodiments illustrated in the drawings, wherein identical reference numerals designate the same components. Note that the features illustrated in the drawings are not necessarily drawn to scale.

FIG. 1 is a block diagram representing one embodiment of a coil assessment system.

FIG. 2 is an image of one example of superimposed unaligned coil masses from two images.

FIG. 3 is an image of one example of superimposed aligned coil masses from two images.

FIG. 4A is an image depicting one example of one or more coil metrics for two images of a coil mass.

FIG. 4B is an image depicting one example of one or more coil metrics for two images of a coil mass.

FIG. 5 is a flow diagram depicting one embodiment of a method for determining a rule set.

FIG. 6 is a flow diagram depicting one embodiment of a method for automated assessment of coil stability using a rule set.

FIGS. 7A-7D are depictions of interfaces that may be utilized by embodiments of a coil assessment system.

### DETAILED DESCRIPTION

The invention and the various features and advantageous details thereof are explained more fully with reference to the nonlimiting embodiments that are illustrated in the accompanying drawings and detailed in the following description. Descriptions of well known starting materials, processing techniques, components and equipment are omitted so as not to unnecessarily obscure the invention in detail. It should be understood, however, that the detailed description and the specific examples, while indicating preferred embodiments of the invention, are given by way of illustration only and not by way of limitation. Various substitutions, modifications, additions and/or rearrangements within the spirit and/or scope of the underlying inventive concept will become apparent to those skilled in the art from this disclosure.

Before discussing specific embodiments, a brief discussion of context particularly with respect to aneurysms and their treatment may be helpful. As discussed previously, the treatment of brain aneurysms using endovascular coiling has grown significantly over the past decade and is now utilized as the treatment option in the majority of cases now.

The use of coiling is not infallible, however, as an aneurysm reoccurs in approximately 16-40% of cases treated using coiling, where such reoccurrences require re-treatment in about 10-20% of cases. Because the risk of aneurysm recurrence after endovascular coiling is higher than surgical clipping, all patients with coiled aneurysms are usually advised to return after some time period (e.g., 6, 12, or 24 months) for a diagnostic evaluation to monitor for a residual or recurring aneurysm. The current standard to ensure treatment durability, and to prevent devastating cerebral hemorrhage from recurrence, is catheter-based angiography. However, follow up with catheter angiography is both invasive (risking clinically significant embolic strokes, hematomas and vascular injury) and expensive, usually requiring serial studies costing thousands of dollars.

Alternatives to evaluation using catheter based angiography have included computerized tomography (CT) or magnetic resonance (MR) based angiography, but these techniques are usually inaccurate due to the presence of metallic coil mass artifacts and difficulty in assessing for recurrence measuring less than about 3 mm. Furthermore, CT or MR angiography is expensive and not available to all patient populations. While in certain cases, x-rays have been used by doctors to assess the stability of endovascular coils, this assessment is performed in a manual and individually subjective assessment by that doctor based on that particular doctor's experience and knowledge.

It would thus be desirable to have automated systems and methods for assessing the stability of endovascular coils using relatively inexpensive and readily available imaging techniques such as x-rays or the like.

To that end, among others, embodiments of systems and methods for rules based assessment of endovascular coil stability are disclosed herein. Embodiments of these systems and methods may assess images from a patient taken at a first time (e.g., at the time of initial coil placement or thereafter) and at one or more second times (e.g., at 6, 12 or 24 months from coil placement) using a set of rules. Based on this assessment, a recurrence parameter or indicator (e.g., a yes or no, a likelihood or probability of recurrence, etc.) may be determined and presented to a user of the system (such as a doctor or other clinician). This recurrence parameter, predictor or indicator (used interchangeably herein) can then be utilized to determine if further procedures should be undertaken to diagnose or treat the patient.

As a particular advantage to embodiments of these systems and methods, the images used for assessment need not necessarily be from a catheter angiogram, a CT scan, or an MR scan. Instead, a simple and usually readily available and relatively inexpensive x-ray may be utilized for one or both the images at the first and the second point in time.

In this way, the x-ray comparison and associated recurrence indictor will potentially be able to prevent the need for a follow up angiogram in certain patients, eliminating the risks or disadvantages associated with any catheter angiogram procedure, which include embolic strokes, hematomas, vascular injury, and cost (e.g., angiograms are expensive averaging about $3,500 a procedure versus a $100 x-ray).

The rules utilized by embodiments of the systems and methods may be generated based on an analysis of previously obtained patient data. In particular, a corpus of patient data may include a set of patient records for patients who have had a coiling procedure performed. These patient records each include a set of images (e.g., x-rays) for that patient (e.g., taken at least at a first and second time) and a recurrence indicator indicating whether that patient has a recurrence of their aneurysm subsequently to having the coiling procedure. Moreover, in some embodiments, the recurrence indicator may be temporally aligned with one of the images for that patient (e.g., the indicator may indicate whether that patient has a recurrence of their aneurysm in close temporal proximity to the time at which the image was taken).

This patient data can be analyzed to generate the rule set to be utilized. Specifically, in certain embodiments one or more metrics of the coil may be determined for each patient in the corpus (or a subset of those patients that may comprise a training data set or the like) based on the set of images for that patient. These coil metrics may include metrics related to properties of the coil such as density or dimensions (e.g., length, width or area) of the coil or the coils relation to other structures (e.g., its relation to bony structures of the body). Moreover, these coil metrics may be related to properties that can be determined about coil from a single image (e.g., from an image taken at a first time or from an image taken at a second time), or may include metrics related to a change of a particular metric over time (e.g., between the image taken at the first time and the image taken at a second time). These coil metrics determined for each of the patients may be utilized along with the recurrence indicator for that patient to determine the rule set utilized by embodiments of the systems and methods as disclosed.

In certain embodiments, this rule set may take the form of, or be embodied in, a machine learning (ML) model such as a logistic regression or a logit model. Embodiments of this logit model may have, for example, the recurrence indicator (e.g., whether the patient had (or will have) a recurrence of the aneurysm) as a binary dependent variable and include predictor variables related to the one or more metrics of the coil in the images of the patient at a first or second point in time. Other embodiments may utilize other types of rules sets such as Random Forest, Rotation Forest, XGBoost, Decision Tree, Gradient Boosting, Multilayer Perception, Discriminant Analysis, Vector Models, Naïve Bayes, Probit Model, or the like.

Thus, the automated rules based assessment of coil stability may allow the simultaneous consideration of multiple variables including, for example, the dimensions (length, width or area) and density of the coil when generating a single metric (or score) predictive or indicative of recurrence (e.g., a predictive recurrence indicator). Additionally, the analysis may be automated, requiring no clinician involvement. Moreover, embodiments of these systems and methods may also be continually improved through the generation of new rules based on better or additional patient data.

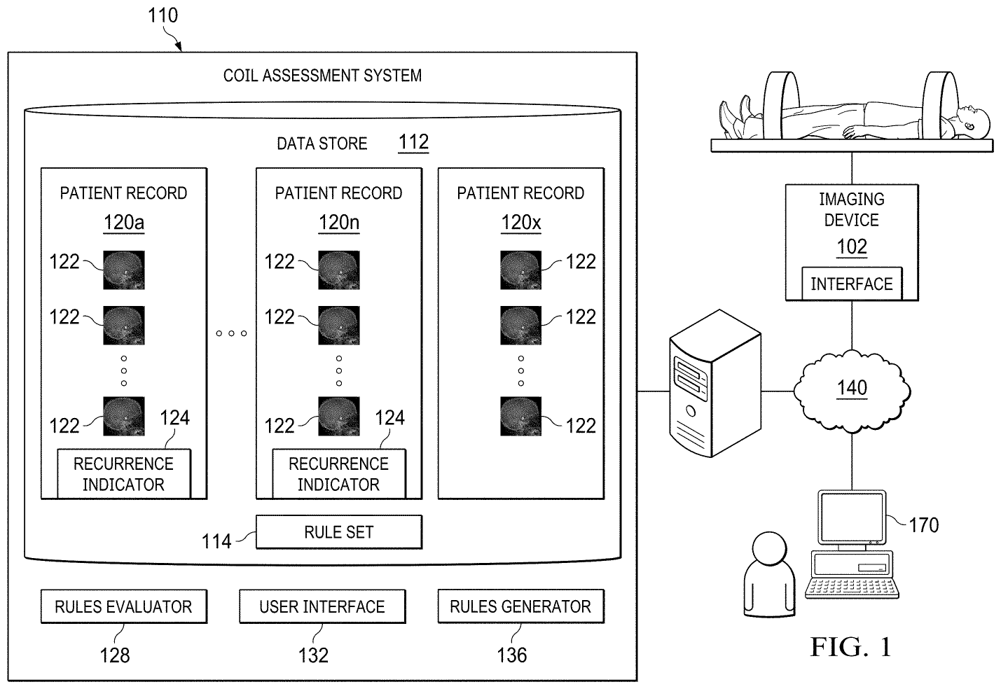

Referring now to FIG. 1, one embodiment of a coil assessment system 110 is depicted. A coil assessment system 110 is a computer system including at least a processor and a computer readable memory including a data store 112. The data store 112 includes a set of patient records 120 for patients that have undergone an aneurysm coiling procedure. Each of the patient records 120 includes a set of images 122 that include the aneurysm coil for that patient. These images 122 may, for example, be in the Digital Imaging and Communications in Medicine (DICOM) format. Each of images 122 for a patient may be from different points in time, including for example a first time (e.g., when the coil was initially placed in the aneurysm of the patient) and a second time (e.g., at around 6 months, 12 months or 24 months after the initial treatment). In one embodiment, each image 122 may include an anterior-posterior skull x-ray or a lateral skull x-ray.

Additionally, a set of the patient records 120 (e.g., here 120a-120n) includes a recurrence indicator 124 indicating whether the patient associated with the patient records 120 had a recurrence of the aneurysm. In one embodiment, a time passage between initial placement of the coil (e.g., when the coiling procedure on the patient was performed) and the time of aneurysm recurrence may also be associated with the patient record 120. The recurrence indicator 124 can, for example, be based on a detailed analysis of a detailed image of the patient or aneurysm such as an angiogram or the like.

The computer readable memory of the coil assessment system may include instructions for execution on the processor of the coil assessment system 110, for a rules generator 136, a rules evaluator 128 and a user interface 132. The rules generator 136 evaluates the set of patient records 120a-120n that includes a recurrence indicator 124 (or a subset thereof) to generate a rule set 114 to store in the data store 112. The rule set 114 may be based on metrics of the coil in the images 122 at a first and second point in time. In order to more effectively compare the coil mass between the images 122 for a patient, then, the rules generator 136 may first align the coil mass identified in each of the images for a patient before determining the coil metrics. Embodiments of such an alignment are described elsewhere herein.

In one embodiment, this rule set 114 may take the form of, or be embodied in, a ML model trained based on a training set comprising at least a subset of patient records 120 that include a recurrence indicator 124, including for example, a logistic regression or a logit model. This logit model may have, for example, the recurrence indicator 124 as a binary dependent variable and include predictor variables related to one or more metrics of the coil in the images 122 at a first and second point in time such as dimensions, density or other metrics derived from each image 122 or a combination of the images 122, or one or more metrics related to time (e.g., the time between the first and second points in time of the images, a time between the coiling procedure and aneurysm recurrence, etc.). In such embodiments, rules generator 136 may train a ML model based on the training set and store the resulting ML model as the rules set 114. The generation of the rule set 114 will be discussed in more detail at a later point herein.

For example, in one embodiment, the rules generator 136 may determine a difference in length of the coil mass between a length of a coil mass as determined from a first AP x-ray image 122 of the patient's skull at a first time and a length of the coil mass as determined from a second AP x-ray image 122 of the patient's skull at a second time. Similarly, a difference in length from a first lateral x-ray image 122 of the patient's skull at a first time and a length of the coil mass as determined from a lateral AP x-ray image 122 at a second time may also be determined. The rules generator 136 can also determine a difference in width between the two AP x-ray images, a difference in width between the two lateral x-ray images, a difference in density between the two AP x-ray images or a difference in density between the two lateral x-ray images. These differences may be utilized as coil metrics (e.g., and thus as predictor variables in an ML model for a rules set 114).

It will be noted at this point that while particular embodiments may utilize coil metrics determined from, or related to, a single image or from a combination of two images taken at different points in time, other coil metrics pertaining to multiple (e.g., including more than two) image may be utilized in some embodiments without loss of generality, and all such embodiments are contemplated herein.

Rules evaluator 128 may utilize the rule set 114 to determine a (e.g., predictive) recurrence indicator for a patient based on an image for that patient and one or more stored images of that patient from a previous point in time. In particular, in one embodiment, a new image 122 (such as an x-ray including an anterior-posterior image or a lateral image) may be taken of the patient (e.g., of the coil or skull of the patient) by an imaging device 102 and communicated to the coil assessment system 110 through a computer network 140 such as a Local Area Network (LAN), a Wide Area Network (WAN), the Internet, an intranet, a cellular network, a wireless computer network, some combination of networks, etc. It will also be apparent that such an image 122 may not be a “new” image but an image taken at almost any point in time subsequently to a stored image 122 for the patient from a previous point in time and may be provided and stored at the coil assessment system 110 in a variety of other manners including receiving the image 122 from another source (e.g., over network 140) or provided by a user from a computer readable medium, etc.).

A user (e.g., doctor, x-ray technologist, radiologist, etc.) at a computer device 170 accessing a user interface 132 of the coil assessment system 110 may indicate that the new image 122 is associated with a patient having a patient record 120x including one or more images 122 from a previous point in time (e.g., when the coil of the patient was first placed) for that patient. This new image 122 can then be stored in the patient record 120x.

Moreover, the user at the computer device 170 may indicate through the user interface 132 that a (e.g., predictive) recurrence indicator is to be generated for that patient (e.g., the patient associated with patient record 122x). The user interface 132 may invoke the rules evaluator 128 with an identification of the patient for which the predictive recurrence indicator is to be generated. The rules evaluator 128 may then obtain two images 122 at two different points in time for the identified patient from the patient record 120 for the identified patient.

For example, an image 122 of the patient from the time of initial treatment of the patient may be automatically utilized by the coil assessment system 110 as an image from a first time and the most recent image 122 of the patient may be utilized by the rules evaluator 128 as the image from a second time. As another possibility, the two most recent images 122 may be automatically selected for use by the rules evaluator 128. Alternatively, in one embodiment, the user at the computing device 170 may use the user interface 132 to select two or more images 122 of the patient from different points in time that are to be utilized to generate the predictive recurrence parameter for the patient.

The two images for the patient (e.g., an anterior-posterior (AP) x-ray image and a lateral x-ray image from a first time and an AP x-ray image and lateral x-ray image from a second time) may be presented to the user at the computer device 170 using the user interface 132. The user interface 132 may then request the user of the coil assessment system 110 to identify the coil in each of the images presented on the user interface. Alternatively, in one embodiment, image analysis techniques may be utilized to identify the coil in each of the selected images for the patient.

Based on the coil identification in the images, the rules evaluator 128 then determine one or more coil metrics for the identified coil. These coil metrics may include, for example, dimensional metrics such as length, width, area, density, distribution or orientation. In particular, in one embodiment, the rules evaluator may take each image from the first time (e.g., an AP x-ray image or lateral x-ray image of the patient's skull) and compare it with a corresponding image from the second time (e.g., the corresponding AP x-ray image or lateral x-ray image of the patient's skull).

To compare the corresponding images from the two times the rules evaluator 128 may first align the identified coil (also referred to as the coil mass) in each of the selected images for the patient. It will be apparent to those of skill in the art that two images of the same patient taken at two different times may differ in dimensionality or orientation because of variables such as patient orientation at the time of imaging (e.g., the tilt of the patient's head, etc.), the type of machine on which each image was taken, or other variables.

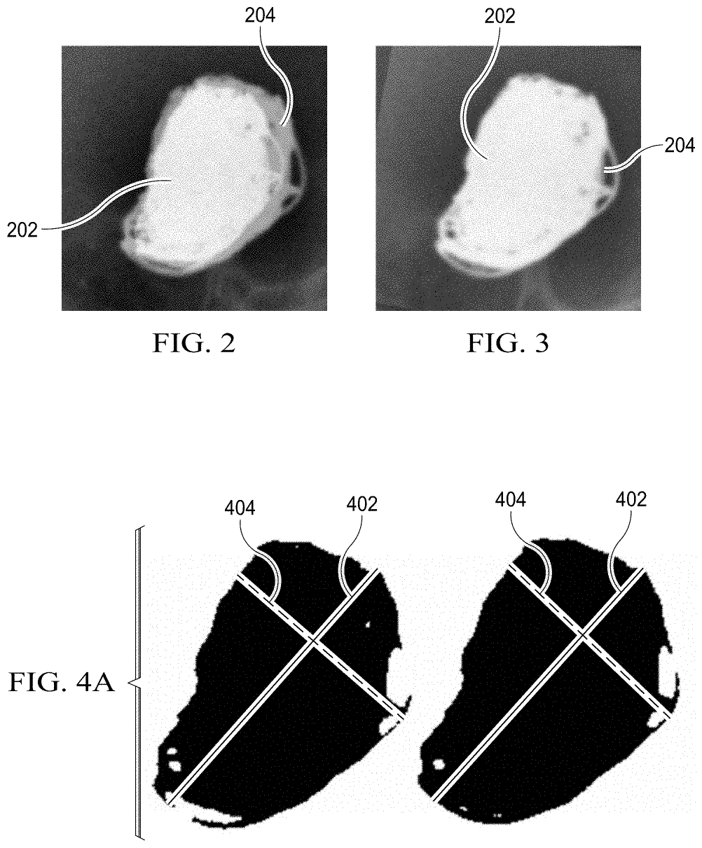

A depiction of two different images of the same coil mass superimposed without alignment is depicted in FIG. 2. AS can be realized from looking at coil mass 202 and coil mass 204, it may be difficult to compare (e.g., determine equivalent coil metrics for) coil masses 202, 204 from different images (or determine coil metrics from these images) when the coil masses 202, 204 are misaligned.

Returning to FIG. 1, in order to more effectively compare the coil mass between the images 122 for a patient, then, the rules evaluator 128 may first segment re-align the coil mass identified in each of the images. The alignment may be accomplished using, for example, image registration or alignment tools available in MATLAB. A depiction of two different images of the same coil mass 202, 204 superimposed after alignment is depicted in FIG. 3.

Moving back to FIG. 1, once the coil masses identified in each image from the respective time periods have been segmented or aligned, the set of coil metrics may be determined. In one embodiment, a length and a width may be determined for the coil mass in each selected image. To determine a length for the identified coil mass in an image each pixel of the coil mass in the image may be compared to each other pixel of the identified coil mass to determine a distance between each pair of pixels of the coil mass. The pair of pixels having the longest distance between then may be considered the pair of pixels defining a line along the length of the coil mass, with the distance between them being the length of the coil mass.

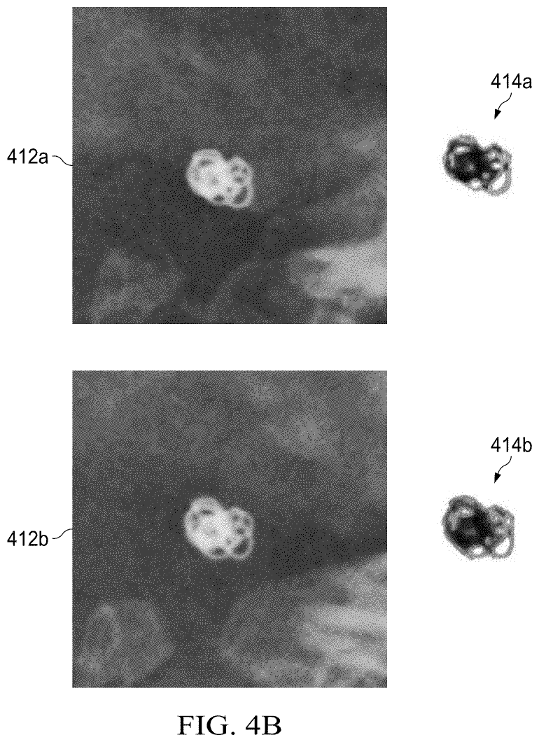

To determine a width for the coil mass, each pair of pixels defining a line approximately perpendicular to the length may be evaluated to determine the pair of pixels that define the longest line that is approximately perpendicular to the length. The length of this line is considered the width for the coil mass of the image. A graphical depiction of the length 402 and width 404 of two images of the same coil mass from two different points in time is depicted in FIG. 4A.

In one embodiment, a density or density map (e.g., a heat map) of the coil mass of the image (or a difference between the density of the coil mass in the first image and second image) may also be determined as a coil metric. This density or density map may be determined using Hounsfield units reported or derived from the data associated with the image 122 (e.g., an image in DICOM format). One or more coil metrics may be determined by comparing the density or density map determined from each image. FIG. 4B depicts a coil mass from a first image 412a and a related density map 414a (e.g., shown as a heat map) and the coil mass from a second image 412b and a related density map 414b (e.g., shown as a heat map).

Looking back at FIG. 1, thus, for each image 122 from the first time (e.g., an AP x-ray image or lateral x-ray image of the patient's skull) a set of coil metrics may be determined. Similarly, for each image from the second time (e.g., an AP x-ray image or lateral x-ray image of the patient's skull) a corresponding set of coil metrics may also be determined. The rules evaluator 128 may utilize the first coil metrics from the images from the first time and the second coil metrics determined from the images from the second time to determine a recurrence indicator by applying the rules set 114 (e.g., as embodied in an ML model) stored in the data store 112.

Specifically, in one embodiment the rule set 114 may take the form of a logistic regression or a logit model. This logit model may have, for example, the recurrence indicator as a binary dependent variable and include predictor variables related to one or more coil metrics. In particular, in one embodiment, the predictor variables may include a difference in the length of the coil mass in an image from a first time (e.g., an AP x-ray image or lateral x-ray image of the patient's skull) and the length of the coil mass in the corresponding image (e.g., an AP x-ray image or lateral x-ray image of the patient's skull) from the second time; a difference in the width of the coil mass in the image from a first time and the width of the coil mass in the corresponding image from the second time; or a difference in the density of the coil mass in the image from a first time and the density of the coil mass in the corresponding image from the second time. It will be apparent that other coil metrics and predictor variables may be utilized and are fully contemplated herein.

The rules evaluator 128 can then determine a value for the predictor variables of the rules set 114 using the coil metrics determined for each of the images associated with patient 122. For example, in one embodiment, the rules evaluator 128 may determine a difference in length of the coil mass between a length of a coil mass as determined from a first AP x-ray image 122 of the patient's skull at a first time and a length of the coil mass as determined from a second AP x-ray image 122 of the patient's skull at a second time.

Similarly, a difference in length from a first lateral x-ray image 122 of the patient's skull at a first time and a length of the coil mass as determined from a lateral AP x-ray image 122 at a second time may also be determined. The rules evaluator 128 can also determine a difference in width between the two AP x-ray images 122, a difference in width between the two lateral x-ray images 122, a difference in density between the two AP x-ray images 122 or a difference in density between the two lateral x-ray images 122.

The rule set 114 may be applied to the determined values for the predictor variables for the patient as determined from the analysis of images 122 of the patient from the two points in time to generate a predictive recurrence indicator (e.g., a binary or other type of value predictive of whether the recurrence of the aneurysm will occur). This predictive recurrence indicator may be returned to the user at the computer device 170 using the user interface 132. The recurrence indicator can thus be reported to a clinician who can make a determination based on this recurrence indicator if further diagnostics or evaluations related to the patient's aneurysm or coil should be undertaken.

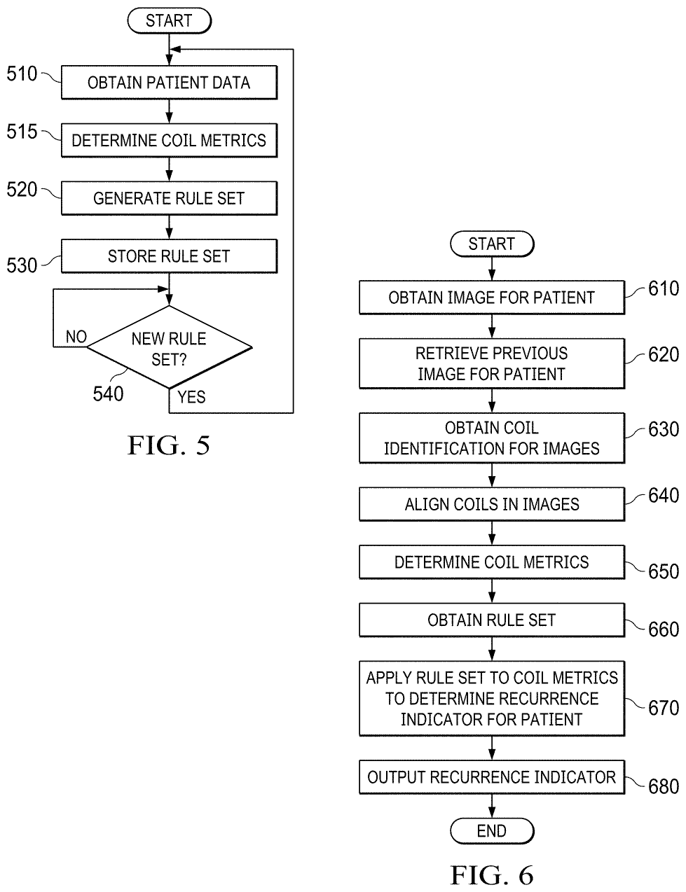

Turning now to FIG. 5, a flow diagram for one embodiment of a method for generating a rule set for use by a coil assessment system is depicted. Initially, patient data is obtained by the system (STEP 510). As discussed, this patient data may include data for a set of patients that have undergone an aneurysm coiling procedure. The patient data for each patient thus includes at least a first image (e.g., an AP x-ray of the patient's skull or a lateral x-ray of the patient's skull) for the patient obtained at a first time, such as when the aneurysm coiling procedure was initially performed on the patient, and a second image (e.g., an AP x-ray of the patient's skull or a lateral x-ray of the patient's skull) for the patient obtained at a second time, such as 6 months, 12 months or 24 months after the coiling procedure was performed on the patient. The patient data for each patient may also include a recurrence indicator indicating whether the associated patient had a recurrence of the aneurysm. Moreover, in some embodiments, the recurrence indicator may be temporally aligned with one of the images for that patient (e.g., the indicator may indicate whether that patient has a recurrence of their aneurysm in close temporal proximity to the time at which the image was taken).

Using this patient data a rule set may be generated. To generate the rule set, values for one or more desired coil metrics (e.g., difference in length, difference in width, difference in density or the like) may be determined based on the images associated with each patient represented in the patient data (STEP 515). Thus, for the patient data pertaining to a particular patient, the images may be analyzed substantially to generate the desired coil metrics for that patient.

These coil metrics may include, for example, a difference in length of the coil mass between a length of a coil mass as determined from a first AP x-ray image of the patient's skull at a first time; a length of the coil mass as determined from a second AP x-ray image of the patient's skull at a second time; a difference in length from a first lateral x-ray image of the patient's skull at a first time and a length of the coil mass as determined from a lateral AP x-ray image at a second time; a difference in width between two AP x-ray images; a difference in width between the two lateral x-ray images; a difference in density between the two AP x-ray images; or a difference in density between the two lateral x-ray images.

The coil metrics (e.g., difference in length, difference in width, difference in density, etc.) in association with the recurrence indicator for that patient may be used to generate the rule set for subsequent determination of recurrence indicators for patients (STEP 520). In one embodiment, this rule set may take the form or, or be embodied in, a ML model such as a logistic regression model. Embodiments of this logit model may have, for example, the recurrence indicator (e.g., whether the patient had (or will have) a recurrence of the aneurysm) as a binary dependent variable and include predictor variables related to the one or more coil metrics derived from the images of the patient at a first or second point in time. such as dimensions, density or other metrics derived from each image or a combination of the images, or one or more metrics related to time (e.g., the time between the first and second points in time of the images, a time between the coiling procedure and aneurysm recurrence, etc.).

The model may thus be trained using the coil metrics in association with the recurrence indicator for that patient to, for example, generate appropriate weights for the predictor variables in the model. Other embodiments of rules sets may utilize other types of models such as Random Forest, Rotation Forest, Decision Tree, Gradient Boosting, Multilayer Perception, Discriminant Analysis, Vector Models, Naïve Bayes, Probit Model, or the like.

Once the rule set is generated, it can be stored for later use by the coil assessment system (STEP 530). At some point in the future, such as when new or additional patient data is received or patient data is otherwise updated, a new rule set may be generated (STEP 540). In one embodiment, a new rule set may be generated automatically based on a time interval, such as nightly, weekly or monthly or may be automatically generated based on a threshold amount of new or update patient data. Other triggers for the generation of a new rule set are possible and are fully contemplated herein. It will be understood, then, that embodiments as utilized herein may be equally effectively utilized with supervised, unsupervised or semi-supervised ML models at various points during operation of a coil assessment system. For example, an ML model may be trained initially in a supervised manner but may later be update or re-trained in a semi-supervised or unsupervised manner, such that techniques as active learning or continuous active learning may be effectively utilized in particular embodiments.

Moving on to FIG. 6, a flow diagram for one embodiment for the automated assessment of aneurysm coil stability by a coil assessment system using a set of rules is depicted. Initially, an image for a patient is obtained. This image may be provided or received from an imaging device, input by a user or obtained in some other manner (STEP 610). This image may include an AP x-ray image or a lateral x-ray image of a skull of a patient at a point in time. The point in time may be, for example, when an aneurysm coiling patient comes in for a follow up evaluation at 6, 12 or 24 months, or from some other time subsequent to the performance of an aneurysm coiling procedure on the patient.

A second image for the patient from a previous point in time (e.g., previous to when the first image was taken) may then be obtained (STEP 620). This second image may include, for example, an AP x-ray image or a lateral x-ray image of the skull of the patient taken at a previous point in time, such as when an aneurysm coiling procedure was initially performed on the patient. This second image may have been previously stored or may be provided by a user at a time when an assessment of coil stability is to be performed or may be provided in some other manner.

The two images for the patient (e.g., an anterior-posterior (AP) x-ray image from a first time or a lateral x-ray image from the first time and an AP x-ray image or lateral x-ray image from a second time) may be have a coil mass identified in each of the images (STEP 630). Such an identification of the coil mass in each image may be done in automated manner through image processing techniques.

Alternatively, the images may be presented to a user for identification of the coil mass in each image. It will be noted that this identification of the coil mass may have occurred previously (e.g., in an automated manner or manually by a user, including another user) and be stored with an image. In particular, for user identification of a coil mass the images may be displayed on a user interface of a coil assessment system and a user requested to identify the coil in each of the images presented on the user interface.

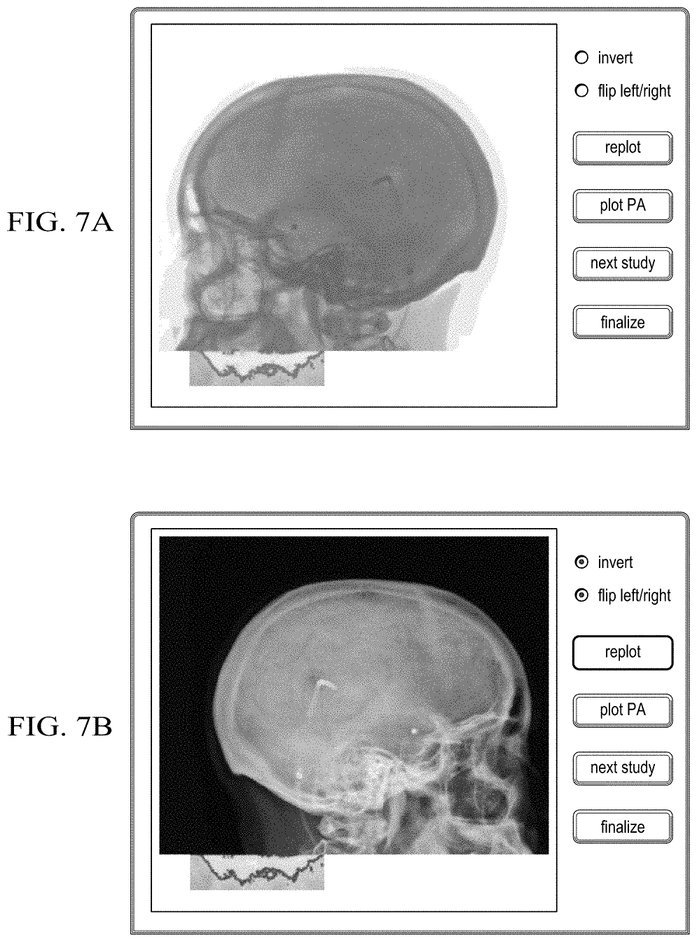

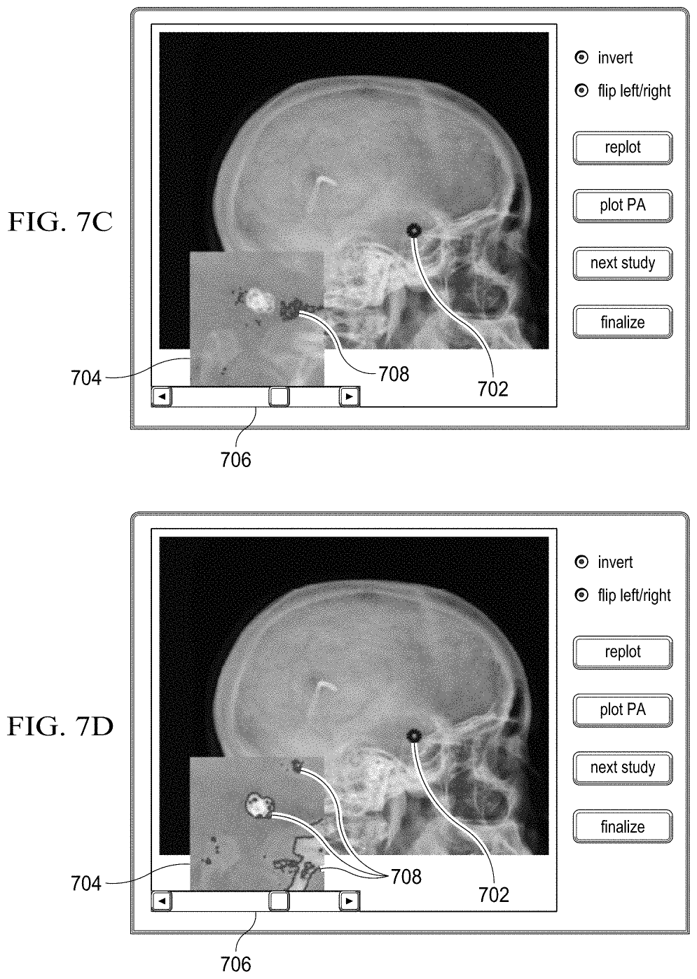

It may be useful to present embodiments of interfaces that may be used for image alignment or coil identification. Referring briefly to FIGS. 7A-7D depictions of interfaces that may be utilized by embodiments of a coil assessment system are presented. In FIGS. 7A and 7B, one embodiment of an interface displaying an x-ray image (e.g., a DICOM image) in a viewer is depicted. The imaging may be standardized (e.g., black as background, facing the right). An option to change the color scale may be selected using the radio-button “invert”, and the orientation may be changed with the radio button “flip left/right”.

Looking at FIGS. 7C and 7D, a user can click on the image in the vicinity of the coil and the interface will plot a (e.g., colored) circle 702 over the click location and then plot the data as an inset 704 in the lower left. Using the slide bar 706, the user can threshold to get the correct level of the coil density. Sliding right may indicate higher density. Lines 708 (e.g., which may be colored for distinctness) show which data is above/below that density threshold (e.g., selected using slider 706). Once all image studies and AP/Lateral have been processed, the user clicks the “finalize” button and the coil locations and thresholds are saved with the images.

Returning to FIG. 6, to compare the corresponding images from the two times the identified coil mass in the images may be aligned (STEP 640). It will be apparent to those of skill in the art that two images of the same patient taken at two different times may differ in dimensionality and orientation because of variables such as patient orientation at the time of imaging (e.g., the tilt of the patient's head, etc.). In order more effectively compare the coil mass between the images, then, the coil mass in each corresponding image from each time may be segmented or re-aligned. For example, the coil mass identified in each AP x-ray from the two times may be aligned and the coil mass identified in each lateral x-ray from the two times may be aligned. The alignment may be accomplished using, for example, image registration or alignment tools available in MATLAB.

Once the coil mass has been aligned in the images, one or more metrics for the coil may be determined (STEP 650). These coil metrics may include, for example, dimensional metrics such as length, width, area, density, distribution or orientation. In particular, in one embodiment, each image (e.g., the coil mass as identified in each image) from the first time (e.g., an AP x-ray image or lateral x-ray image of the patient's skull) and compare it with (the coil mass as identified in) the corresponding image from the second time (e.g., the corresponding AP x-ray image or lateral x-ray image of the patient's skull).

In one embodiment, a length and a width may be determined for the coil mass in each image. To determine a length for the identified coil mass in an image each pixel of the coil mass in the image may be compared to each other pixel of the identified coil mass to determine a distance between each pair of pixels of the coil mass. The pair of pixels having the longest distance between then may be considered the pair of pixels defining a line along the length of the coil mass, with the distance between them being the length of the coil mass.

To determine a width for the coil mass, each pair of pixels defining a line approximately perpendicular to the length may be evaluated to determine the pair of pixels that define the longest line that is approximately perpendicular to the length. The length of this line is considered the width for the coil mass of the image.

A density or density map (e.g., as represented in a heat map) of the coil mass of the image may also be determined as a coil metric. This density or density map may be determined using Hounsfield units reported or derived from the data associated with the image (e.g., an image in DICOM format). One or more coil metrics may be determined by comparing the density or density map determined from each image.

Thus, for each image from the first time (e.g., an AP x-ray image or lateral x-ray image of the patient's skull) a set of coil metrics may be determined. Similarly, for each image from the second time (e.g., an AP x-ray image or lateral x-ray image of the patient's skull) a corresponding set of coil metrics may also be determined. In one embodiment, these coil metrics can include, for example, a difference in length of the coil mass between a length of a coil mass as determined from a first AP x-ray image of the patient's skull at a first time and a length of the coil mass as determined from a second AP x-ray image of the patient's skull at a second time. Similarly, a difference in length from a first lateral x-ray image of the patient's skull at a first time and a length of the coil mass as determined from a lateral AP x-ray image at a second time may also be used as coil metric. A difference in width between the two AP x-ray images, a difference in width between the two lateral x-ray images, a difference in density between the two AP x-ray images or a difference in density between the two lateral x-ray images may also be utilized as coil metrics. Again, it will be noted that while particular embodiments may utilize coil metrics determined from, or related to, a single image or from a combination of two images taken at different points in time, other coil metrics pertaining to multiple (e.g., including more than two) image may be utilized in some embodiments without loss of generality.

In one embodiment, the coils of the images may be found and coil metrics determined according to an embodiment as defined by the following pseudocode:

find_coil.m
input: raw DICOM image data, coil threshold, field of view for
analysis
initialize output matrix (coil_vox) as all 0's
initialize binary matrix with 0's for sub-threshold and 1's
for supra-threshold pixels (threshold_vox)
use user designated “coil center” to start analysis

if this pixel is not supra-threshold, find nearest
supra-threshold pixel to center analysis

set center of analysis in coil_vox as 1 (included in the coil)
and initialize list of pixels in coil with this pixel only
while list of possible pixels is not empty

find first pixel that has not been analyzed
add one in coil_vox to 1 for all pixels adjacent to that
pixel
set coil_vox equal to dot-multiply of coil_vox by
threshold_vox
add all new non-zero pixels in coil_vox to the list of
possible pixels
terminate when no new pixels are added and all pixels
within/next to coil have been analyzed

return
coil_dim.m
input: coil_vox from find_coil.m
for coil of n pixels, initialized adjacency matrix n X n as
zeros
for coil of n pixels, initialized angle matrix n X n as zeros
for ii = 1 ... n

for jj = 1...n
compute Euclidean distance pixel ii to jj, store in
adjacency matrix
for that vector of pixel ii to jj, find angle relative
to horizontal and store in angle matrix

find largest difference between pixels in adjacency matrix,
define this value as length
find all lines between pixels that are perpendicular to length

using the angle matrix, lines between pixels that are
within a defined tolerance of orthogonal are identified

using adjacency matrix, find longest orthogonal line, define
this length as width
return
coil_process.m
load raw DICOM data
align follow up datasets to the baseline x-ray or angiographic
AP/lateral films

6-degree affine transform, maximum iterations 300,
optimize for similarity using imregister.m

run find_coil.m
run coil_dim.m
for coil density correlation, find overlap of coil pixels in
baseline and follow up study

compute Pearson's correlation for coil density at common
pixels
save rho and p-value

output the baseline length, width and area
output the follow up length width, area and correlation
run R logistic regression prediction using model from prior
data
logistic regression returns a 1 for change in coil or 0 for no
change
return

Accordingly, the first coil metrics from the images from the first time and the second coil metrics determined from the images from the second time (or coil metrics resulting from a difference or other comparisons between coil metrics derived from the images) may be used to determine a recurrence indicator by applying a rule set.

Thus, a rules set may be obtained (STEP 660) and applied to the coil metrics determined from the patient's images to determine a recurrence parameter for the patient (STEP 670). Specifically, in one embodiment the rule set may take the form of a logistic regression or a logit model. This logit model may have a, for example, the recurrence indicator as a binary dependent variable and include predictor variables related to one or more coil metrics. Other embodiments may utilize other types of rules sets such as Random Forest, Rotation Forest, XGBoost, Decision Tree, Gradient Boosting, Multilayer Perception, Discriminant Analysis, Vector Models, Naïve Bayes, Probit Model, or the like.

A value for each predictor variables of the rules set may be determined using the coil metrics determined for each of the images associated with patient (STEP 650). The rule set may then be applied to the determined values for the predictor variables (e.g., coil metrics) as determined from the analysis of images of the patient from the two (or more) points in time to generate a predictive recurrence indicator (e.g., a binary or other type of value indicating or predictive of whether recurrence of the aneurysm will occur). This predictive recurrence indicator may be output to a user of a coil assessment system (STEP 680). The predictive recurrence indicator can thus be reported to a clinician who can make a determination based on this predictive recurrence indicator if further diagnostics or evaluations related to the patient's aneurysm or coil should be undertaken.

In the foregoing specification, the invention has been described with reference to specific embodiments. However, one of ordinary skill in the art appreciates that various modifications and changes can be made without departing from the scope of the invention. Accordingly, the specification and figures are to be regarded in an illustrative rather than a restrictive sense, and all such modifications are intended to be included within the scope of invention.

Although the invention has been described with respect to specific embodiments thereof, these embodiments are merely illustrative, and not restrictive of the invention. The description herein of illustrated embodiments of the invention is not intended to be exhaustive or to limit the invention to the precise forms disclosed herein (and in particular, the inclusion of any particular embodiment, feature or function is not intended to limit the scope of the invention to such embodiment, feature or function). Rather, the description is intended to describe illustrative embodiments, features and functions in order to provide a person of ordinary skill in the art context to understand the invention without limiting the invention to any particularly described embodiment, feature or function. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention.

Reference throughout this specification to “one embodiment,” “an embodiment,” or “a specific embodiment” or similar terminology means that a particular feature, structure, or characteristic described in connection with the embodiment is included in at least one embodiment and may not necessarily be present in all embodiments. Thus, respective appearances of the phrases “in one embodiment,” “in an embodiment,” or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

Any suitable programming language can be used to implement the routines, methods or programs of embodiments of the invention described herein, including C, C++, Java, assembly language, etc. Different programming techniques can be employed such as procedural or object oriented. Data may be stored in a single storage medium or distributed through multiple storage mediums, and may reside in a single database or multiple databases (or other data storage techniques

A “computer-readable medium” may be any medium that can contain, store, communicate, propagate, or transport the program for use by or in connection with the instruction execution system, apparatus, system or device. The computer readable medium can be, by way of example, only but not by limitation, an electronic, magnetic, optical, electromagnetic, infrared, or semiconductor system, apparatus, system, device, propagation medium, or computer memory. Such computer-readable medium shall generally be machine readable and include software programming or code that can be human readable (e.g., source code) or machine readable (e.g., object code).

Furthermore, the term “or” as used herein is generally intended to mean “and/or” unless otherwise indicated. As used herein, a term preceded by “a” or “an” (and “the” when antecedent basis is “a” or “an”) includes both singular and plural of such term (i.e., that the reference “a” or “an” clearly indicates only the singular or only the plural). Also, as used in the description herein, the meaning of “in” includes “in” and “on” unless the context clearly dictates otherwise.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component.

What is claimed is:

1. An aneurysm coil assessment system, comprising:
a processor;
a non-transitory, computer-readable storage medium, including computer instructions for:
obtaining patient data on a set of patients, each patient having had an aneurysm coil procedure for an aneurysm, wherein the patient data for a first patient includes a first image of a coil placed in the aneurysm coil procedure for the first patient from a first time and a second image of the coil of the first patient from a second time and a recurrence indicator indicating whether that first patient has a recurrence of their aneurysm subsequently to having the aneurysm coiling procedure;
generating values for a set of coil assessment metrics for each of the set of patients from the patient data for each patient, wherein the values for the set of coil assessment metrics for the first patient is based on the first image of the coil of the first patient from the first time and the second image of the coil of that first patient for the second time;
generating a rule set for generating a predictive recurrence indicator based on the values for the set of coil assessment metrics for each of the set of patients and the recurrence indicator associated with each of the set of patients;
receiving an indication from a user that the predictive recurrence indicator is to be generated for a second patient;
obtaining a first image of a coil placed in an aneurysm coiling procedure for the second patient from a first time and a second image of the coil of the second patient from a second time;
generating values for the set of coil assessment metrics for the second patient based on the first image of the coil of the second patient from the first time and the second image of the coil of that second patient for the second time;
applying the rule set to the values for the set of coil assessment metrics for the second patient to generate the predictive recurrence indicator based on the values for the set of coil assessment metrics for the second patient, wherein the predictive recurrence indicator is predictive of recurrence of the coil in the second patient; and
presenting the predictive recurrence indicator to the user.

2. The system of claim 1, wherein the second image for the second patient is obtained from an imaging device over a computing network.

3. The system of claim 1, wherein the coil assessment metrics include density of the coil, length of the coil, or width of the coil.

4. The system of claim 1, wherein the first image or second image are an anterior-posterior skull x-ray or a lateral skull x-ray.

5. The system of claim 1, wherein the rule set is a logit model.

6. The system of claim 1, wherein generating values for the set of coil assessment metrics for each of the set of patients comprises aligning the first image of the coil of the first patient from the first time with the second image of the coil of that first patient for the second time and generating the values for the set of coil assessment metrics for the second patient comprises aligning the first image of the coil of the second patient from the first time with the second image of the coil of that second patient for the second time.

7. The system of claim 6, wherein aligning the first image and the second image for the first patient comprises aligning the coil in the first image of the first patient with the coil in the second image of the first patient and aligning the first image and the second image for the second patient comprises aligning the coil in the first image of the second patient with the coil in the second image of the second patient.

8. A method for aneurysm coil assessment system, comprising:
obtaining patient data on a set of patients, each patient having had an aneurysm coil procedure for an aneurysm, wherein the patient data for a first patient includes a first image of a coil placed in the aneurysm coil procedure for the first patient from a first time and a second image of the coil of the first patient from a second time and a recurrence indicator indicating whether that first patient has a recurrence of their aneurysm subsequently to having the aneurysm coiling procedure;
generating values for a set of coil assessment metrics for each of the set of patients from the patient data for each patient, wherein the values for the set of coil assessment metrics for the first patient is based on the first image of the coil of the first patient from the first time and the second image of the coil of that first patient for the second time;
generating a rule set for generating a predictive recurrence indicator based on the values for the set of coil assessment metrics for each of the set of patients and the recurrence indicator associated with each of the set of patients;
receiving an indication from a user that the predictive recurrence indicator is to be generated for a second patient;
obtaining a first image of a coil placed in an aneurysm coiling procedure for the second patient from a first time and a second image of the coil of the second patient from a second time;
generating values for the set of coil assessment metrics for the second patient based on the first image of the coil of the second patient from the first time and the second image of the coil of that second patient for the second time;
applying the rule set to the values for the set of coil assessment metrics for the second patient to generate the predictive recurrence indicator based on the values for the set of coil assessment metrics for the second patient, wherein the predictive recurrence indicator is predictive of recurrence of the coil in the second patient; and
presenting the predictive recurrence indicator to the user.

9. The method of claim 8, wherein the second image for the second patient is obtained from an imaging device over a computing network.

10. The method of claim 8, wherein the coil assessment metrics include density of the coil, length of the coil, or width of the coil.

11. The method of claim 8, wherein the first image or second image are an anterior-posterior skull x-ray or a lateral skull x-ray.

12. The method of claim 8, wherein the rule set is a logit model.

13. The method of claim 8, wherein generating values for the set of coil assessment metrics for each of the set of patients comprises aligning the first image of the coil of the first patient from the first time with the second image of the coil of that first patient for the second time and generating the values for the set of coil assessment metrics for the second patient comprises aligning the first image of the coil of the second patient from the first time with the second image of the coil of that second patient for the second time.

14. The method of claim 13, wherein aligning the first image and the second image for the first patient comprises aligning the coil in the first image of the first patient with the coil in the second image of the first patient and aligning the first image and the second image for the second patient comprises aligning the coil in the first image of the second patient with the coil in the second image of the second patient.

15. A non-transitory computer readable medium, comprising instruction for:
obtaining patient data on a set of patients, each patient having had an aneurysm coil procedure for an aneurysm, wherein the patient data for a first patient includes a first image of a coil placed in the aneurysm coil procedure for the first patient from a first time and a second image of the coil of the first patient from a second time and a recurrence indicator indicating whether that first patient has a recurrence of their aneurysm subsequently to having the aneurysm coiling procedure;
generating values for a set of coil assessment metrics for each of the set of patients from the patient data for each patient, wherein the values for the set of coil assessment metrics for the first patient is based on the first image of the coil of the first patient from the first time and the second image of the coil of that first patient for the second time;
generating a rule set for generating a predictive recurrence indicator based on the values for the set of coil assessment metrics for each of the set of patients and the recurrence indicator associated with each of the set of patients;
receiving an indication from a user that the predictive recurrence indicator is to be generated for a second patient;
obtaining a first image of a coil placed in an aneurysm coiling procedure for the second patient from a first time and a second image of the coil of the second patient from a second time;
generating values for the set of coil assessment metrics for the second patient based on the first image of the coil of the second patient from the first time and the second image of the coil of that second patient for the second time;
applying the rule set to the values for the set of coil assessment metrics for the second patient to generate the predictive recurrence indicator based on the values for the set of coil assessment metrics for the second patient, wherein the predictive recurrence indicator is predictive of recurrence of the coil in the second patient; and
presenting the predictive recurrence indicator to the user.

16. The non-transitory computer readable medium of claim 15, wherein the second image for the second patient is obtained from an imaging device over a computing network.

17. The non-transitory computer readable medium of claim 15, wherein the coil assessment metrics include density of the coil, length of the coil, or width of the coil.

18. The non-transitory computer readable medium of claim 15, wherein the first image or second image are an anterior-posterior skull x-ray or a lateral skull x-ray.

19. The non-transitory computer readable medium of claim 15, wherein the rule set is a logit model.

20. The non-transitory computer readable medium of claim 15, wherein generating values for the set of coil assessment metrics for each of the set of patients comprises aligning the first image of the coil of the first patient from the first time with the second image of the coil of that first patient for the second time and generating the values for the set of coil assessment metrics for the second patient comprises aligning the first image of the coil of the second patient from the first time with the second image of the coil of that second patient for the second time.

21. The non-transitory computer readable medium of claim 20, wherein aligning the first image and the second image for the first patient comprises aligning the coil in the first image of the first patient with the coil in the second image of the first patient and aligning the first image and the second image for the second patient comprises aligning the coil in the first image of the second patient with the coil in the second image of the second patient.
